(19) United States
(12) Patent Application Publication
(10) Pub. No.: US 2018/0125910 A1
(43) Pub. Date: May 10, 2018

(54) METHOD OF PRODUCING BEVERAGES ON THE BASIS OF JUICE AND POWDER FROM THE MACA ROOT

(71) Applicant: Kieu Hoang, Westlake Village, CA (US)

(72) Inventor: Kieu Hoang, Westlake Village, CA (US)

(21) Appl. No.: 15/672,151

(22) Filed: Aug. 8, 2017

Related U.S. Application Data
(60) Provisional application No. 62/371,889, filed on Aug. 8, 2016.

(51) Int. Cl. A61K 36/31; A61K 9/00; A23L 2/02; A23L 19/10

(52) U.S. Cl. CPC A61K 36/31; A61K 9/0095; A23V 2002/00; A23L 19/10; A61K 2236/15; A23L 2/02

(57) ABSTRACT

A food composition arranged for human consumption comprising a plurality of ingredients, all from maca root. The invention provides a process for obtaining juice and powder from maca root, wherein the seed of maca root is grinded and centrifuged to make juice and powder. Total 837 protein found in maca root. Among them, 296 are uncharacterized and 35 are putative uncharacterized new found KH good healthy proteins synthesized by KH good healthy cells.

### CROSS-REFERENCE TO EARLIER APPLICATION

This application claims the benefit of U.S. Provisional Application 62/371,889, filed Aug. 8, 2016, which application is incorporated herein by reference.

### SUMMARY OF THE INVENTION

A food composition arranged for human consumption comprising a plurality of ingredients, all from maca root. The invention provides a process for obtaining juice and powder from maca root, wherein the seed of maca root is grinded and centrifuged to make juice and powder. Total 837 protein found in maca root. Among them, 296 are uncharacterized and 35 are putative uncharacterized new found KH good healthy proteins synthesized by KH good healthy cells.

### BRIEF DESCRIPTION OF THE DRAWINGS

FIG. 1 flow chart of maca root process

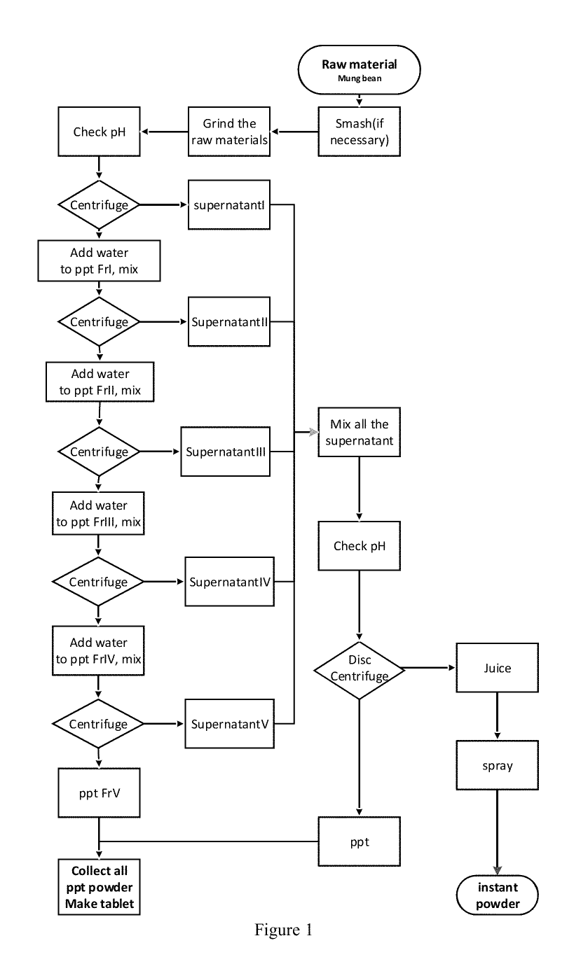

FIG. 2 SDS-Page of supernatant and paste of KH 175 yellow Maca root

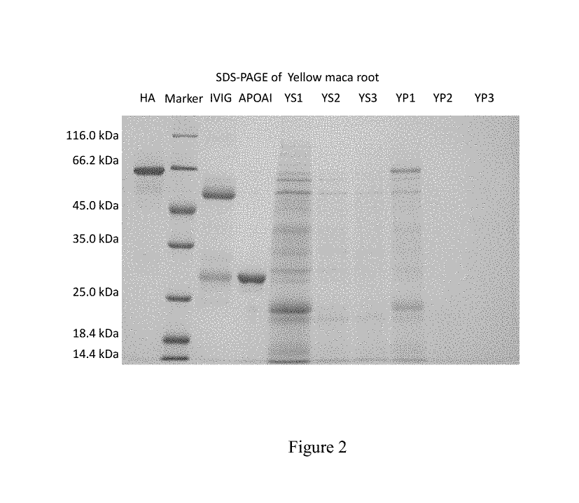

FIG. 3 SDS-Page of supernatant and paste of KH 175 purple Maca root

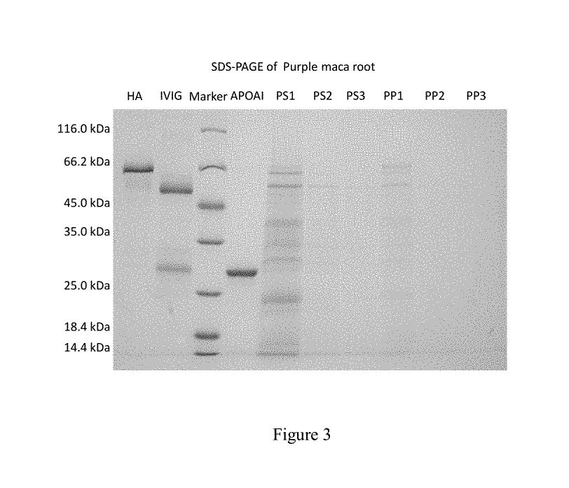

FIG. 4 SDS-Page of supernatant and paste of KH 175 black Maca root

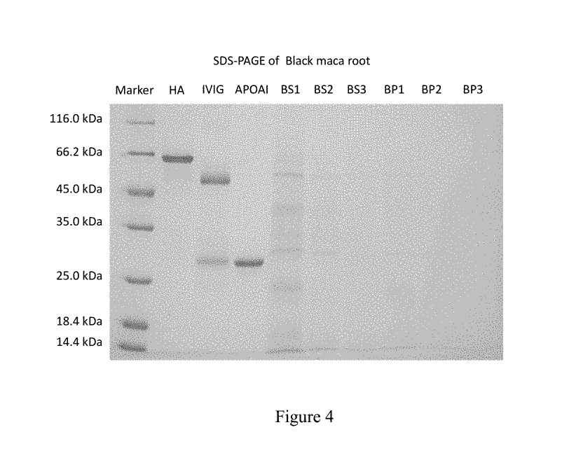

### DETAILED DESCRIPTION OF THE INVENTION

The present invention describes a method for the production of juice and powder from maca root.

The maca root is treated immediately after harvest.

Wash the maca root in cleaning bath at 5 about 0-100 Celsius.

Smash the whole maca root in grinder to cut the thick stem at about 0-100 Celsius. Collect all the raw materials.

Grind the raw materials in superfine mill at about 0-100 Celsius. Collect the raw juice.

Check the pH of the raw juice, about 2.5-10

Centrifuge the raw juice at low to high rpm with normal centrifuge at about −30 to 30 Celsius. Collect the precipitate (“ppt”) and supernatant.

Add 2-4 volume water to ppt. Mix about 10-100 minutes and centrifuge again at low to high rpm with normal centrifuge at about −30 Celsius to 60. Collect the ppt 15 and supernatant.

Add 1-2 volume water to it. Mix about 10-100 minutes and centrifuge again at low to high rpm with normal centrifuge at about −30 to 60 Celsius. Collect the ppt and supernatant.

Mix all the supernatant for 10-100 minutes at about 0-100 Celsius.

Check the supernatant pH, about 2.5 to 10

Centrifuge the supernatant juice at moderate to high rpm with a disc centrifuge at about −30 to 60 Celsius, collect the juice.

Check the juice pH, about.2.5 to 10 pH

### 1. If Making Juice

Homogenizing the juice at 40-60 MPa.
Sterilize the juice at 130-140 Celsius for 5-20 seconds with a pipe sterilizer.
Bottle the clear juice at 25-35 Celsius. Take sample and check the pH

### 2. If Making Powder

The juice is concentrated in Single-effect Falling Film Evaporator at 65-85 Celsius until the brix value is 30-50.
The concentrated juice is transfer to the Centrifugal Spray Dryer to get powder.
Packaging the powder.

### 3. Mix all the 5 Ppt to Make Capsules.

TABLE 1
cell count in the supernatant and paste of KH 175 different maca roots

| | S1 | S2 | S3 | P1 | P2 | P3 |
|---|---|---|---|---|---|---|
| Yellow | 140,000 | 1,240,000 | 513,000 | 442,000,000 | 82,400,000 | 95,200,000 |
| Purple | 146,000 | 553,000 | 111,000 | 189,000,000 | 192,000,000 | 142,000,000 |
| Black | 494,000 | 270,000 | 208,000 | 192,000,000 | 105,000,000 | 145,000,000 |

1. A food composition arranged for human consumption comprising a plurality of ingredients, all from maca root.

2. A method for obtaining juice and powder from maca root, wherein the seed of maca root is grinded and centrifuged to make juice and powder.

3. Method of claim 2, wherein juice is mixed beverage that will contain certain number of cells.

4. Method of claim 2, wherein Powder is Mixed food supplement that will also contain certain number of cells.

5. Method of claim 2, maca root has KH GOOD HEALTHY CELLS in which the RNA synthesizes good proteins that: 1—Send signal to the DAMAGED, SICK, AND BAD CELLS that triggers that synthesis of good proteins that transform these cells to become GOOD healthy cells. 2—Send signal to the other currently undamaged cells to synthesis of good proteins to protect them from being DAMAGED, INFECTED and PRONE to DNA and other cellular alterations. 3—Send signal to the body to produce new cells that are healthy and forbid them from being affected by intra- and extracellular damaging signals.

6. Method of claim 2, in maca root contains proteins whose molecular weight are similar to the molecular weight of the human proteins of human albumin, immunoglobulin and APOA-1.

7. Method of claim 2, living cells are found in this dried powder and beverages.
